(19) United States
(12) Patent Application Publication
(10) Pub. No.: US 2025/0108183 A1
(43) Pub. Date: Apr. 3, 2025

(54) AN APPARATUS AND A METHOD FOR PROVIDING NON-INVASIVE INTERMITTENT POSITIVE PRESSURE VENTILATION

(71) Applicant: NEO AVISHKARS PRIVATE LIMITED, BENGALURU (IN)

(72) Inventor: Sreedhara Malla SADASHIVAPPA, BENGALURU (IN)

(21) Appl. No.: 18/729,186

(22) PCT Filed: Jan. 6, 2023
(86) PCT No.: PCT/IB2023/050097

§ 371 Date: Jul. 16, 2024

(30) Foreign Application Priority Data
Jan. 17, 2022 (IN) 202241002660

(51) Int. Cl. A61M 16/00; A61M 16/10; A61M 16/12; A61M 16/20

(52) U.S. Cl. CPC A61M 16/024; A61M 16/0003; A61M 16/0051; A61M 16/1075; A61M 16/12; A61M 16/204; A61M 16/205; A61M 2016/0027; A61M 2016/0033; A61M 2202/0007; A61M 2202/02; A61M 2205/10; A61M 2205/18; A61M 2205/3334; A61M 2205/3344; A61M 2205/3379; A61M 2230/005; A61M 2230/20

(57) ABSTRACT

An apparatus (10) for providing noninvasive intermittent positive pressure ventilation is provided. The apparatus includes a flow control unit (20) including a first flow meter (30) to provide a first predefined volume of medical air. The flow control unit includes a second flow meter (40) to provide a second predefined volume of the medical air. The apparatus includes an inspiratory unit (50) including a first valve (60) to provide the first predefined volume of the medical air to a patient (80) to assist an inspiration. The inspiratory unit includes a second valve (90) to provide the second predefined volume of the medical air to the patient to assist an expiration. The apparatus includes an expiratory unit (100) to generate a peak inspiratory pressure. The expiratory unit is to generate a peak expiratory pressure. The apparatus includes a control unit (150) to control the peak inspiratory pressure, the peak expiratory pressure thereby providing noninvasive intermittent positive pressure ventilation to the patient.

### EARLIEST PRIORITY DATE

This application claims priority from a Complete patent application filed in India having patent application No. 202241002660, filed on Jan. 17, 2022, and titled “AN APPARATUS AND A METHOD FOR PROVIDING NON-INVASIVE INTERMITTENT POSITIVE PRESSURE VENTILATION”.

### FIELD OF INVENTION

Embodiments of the present disclosure relate to the field of devices used for assisted breathing and more particularly to an apparatus and a method for providing noninvasive intermittent positive pressure ventilation.

### BACKGROUND

Continuous positive airway pressure is a mode of respiratory ventilation used for treating medical conditions such as respiratory distress syndrome, meconium aspiration syndrome, apnea of prematurity, transient tachypnea of newborn, respiratory failure, atelectasis and the like. Provision of the positive airway pressure keeps the airways open, thereby assisting inspiration and expiration of a patient. The positive airway pressure may be provided by non-invasive methods and invasive methods. The non-invasive methods may not require an artificial airway to provide ventilation to the patients. On the contrary, the invasive methods may require the artificial airway to provide ventilation to the patients.

Continuous positive airway pressure machines may be used to provide non-invasive ventilation to the patients. Continuous positive air way pressure provides a single magnitude of positive pressure support during expiration and inspiration. Also, the continuous positive airway pressure machines are subjected to failure. Ventilator driven intermittent positive pressure ventilation may be a next choice upon failure of the continuous positive airway pressure machines. Use of the ventilator may increase cost of treatment and availability of the ventilator may not be ensured whenever required. Also, use of the ventilator may cause ventilator associated pneumonia, air leak syndromes, prolonged hospital stay, lung damage and muscle weakness.

Hence, there is a need for an improved apparatus and a method for providing noninvasive intermittent positive pressure ventilation to address the aforementioned issue(s).

### BRIEF DESCRIPTION

In accordance with an embodiment of the present disclosure, an apparatus for providing noninvasive intermittent positive pressure ventilation is provided. The apparatus includes a flow control unit mechanically coupled to a blender. The flow control unit includes a first flow meter adapted to provide a first predefined volume of medical air supplied by the blender. The flow control unit also includes a second flow meter adapted to provide a second predefined volume of the medical air supplied by the blender. The apparatus also includes an inspiratory unit mechanically coupled to the flow control unit. The inspiratory unit includes a first valve mechanically coupled to the first flowmeter. The first valve is adapted to provide the first predefined volume of the medical air provided by the first flowmeter to a patient through a first outlet to assist an inspiration of the patient. The inspiratory unit also includes a second valve mechanically coupled to the second flowmeter. The second valve is adapted to provide the second predefined volume of the medical air provided by the second flowmeter to the patient through the first outlet to assist an expiration of the patient. The apparatus further includes an expiratory unit mechanically coupled to the inspiratory unit. The expiratory unit is adapted to generate a peak inspiratory pressure during the inspiration of the patient by expelling the first predefined volume of the medical air to a liquid medium at a first depth through a first inlet, and a third valve. The expiratory unit is also adapted generate a peak expiratory pressure during the expiration of the patient by expelling the second predefined volume of the medical air to the liquid medium at a second depth through the first inlet, and a fourth valve. The apparatus further includes a control unit operatively coupled to the expiratory unit and the inspiratory unit. The control unit is adapted to provide one or more control signals to operate the first valve, the second valve, the third valve and the fourth valve to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient.

In accordance with another embodiment of the present disclosure, a method for providing noninvasive intermittent positive pressure ventilation is provided. The method includes providing, by a first flow meter, a first predefined volume of medical air supplied by a blender. The method also includes providing, by a second flow meter, a second predefined volume of the medical air supplied by the blender. The method further includes providing, by a first valve, the first predefined volume of the medical air provided by the first flowmeter to a patient through a first outlet to assist an inspiration of the patient. The method also includes providing, by a second valve, the second predefined volume of the medical air provided by the second flowmeter to the patient through the first outlet to assist an expiration of the patient. The method also includes generating, by an expiratory unit, a peak inspiratory pressure during the inspiration of the patient by expelling the first predefined volume of the medical air to a liquid medium at a first depth through a first inlet, and a third valve. The method also includes generating, by the expiratory unit, a peak expiratory pressure during the expiration of the patient by expelling the second predefined volume of the medical air to the liquid medium at a second depth through the first inlet, and a fourth valve. The method further includes providing, by a control unit, one or more control signals to operate the first valve, the second valve, the third valve and the fourth valve to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic representation of an apparatus for providing noninvasive intermittent positive pressure ventilation in accordance with an embodiment of the present disclosure; and

FIG. 2 is a flow chart representing the steps involved in a method for providing noninvasive intermittent positive pressure ventilation in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to an apparatus and a method for providing noninvasive intermittent positive pressure ventilation. In accordance with an embodiment of the present disclosure, an apparatus and a method for providing noninvasive intermittent positive pressure ventilation is provided. The apparatus includes a flow control unit mechanically coupled to a blender. The flow control unit includes a first flow meter adapted to provide a first predefined volume of medical air supplied by the blender. The flow control unit also includes a second flow meter adapted to provide a second predefined volume of the medical air supplied by the blender. The apparatus also includes an inspiratory unit mechanically coupled to the flow control unit. The inspiratory unit includes a first valve mechanically coupled to the first flowmeter. The first valve is adapted to provide the first predefined volume of the medical air provided by the first flowmeter to a patient through a first outlet to assist an inspiration of the patient. The inspiratory unit also includes a second valve mechanically coupled to the second flowmeter. The second valve is adapted to provide the second predefined volume of the medical air provided by the second flowmeter to the patient through the first outlet to assist an expiration of the patient. The apparatus further includes an expiratory unit mechanically coupled to the inspiratory unit. The expiratory unit is adapted to generate a peak inspiratory pressure during the inspiration of the patient by expelling the first predefined volume of the medical air to a liquid medium at a first depth through a first inlet, and a third valve. The expiratory unit is also adapted generate a peak expiratory pressure during the expiration of the patient by expelling the second predefined volume of the medical air to the liquid medium at a second depth through the first inlet, and a fourth valve. The apparatus further includes a control unit operatively coupled to the expiratory unit and the inspiratory unit. The control unit is adapted to provide one or more control signals to operate the first valve, the second valve, the third valve and the fourth valve to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient.

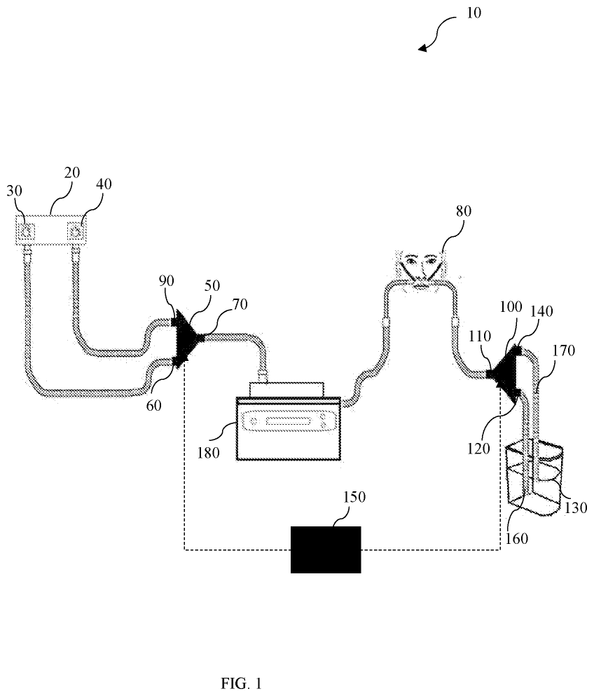

FIG. 1 is a schematic representation of an apparatus (10) for providing noninvasive intermittent positive pressure ventilation in accordance with an embodiment of the present disclosure. The apparatus (10) includes a flow control unit (20) mechanically coupled to a blender (not shown in FIG. 1). As used herein, the blender may be defined as a device which mixes medical air and air in predefined proportions. The flow control unit (20) includes a first flow meter (30) adapted to provide a first predefined volume of medical air supplied by the blender. In one embodiment, the blender may include a motorized blender adapted to provide the medical air to a patient (80) in a concentration range of 21% to 100% through a feedback mechanism based on a blood oxygen concentration of the patient. In detail, the motorized blender may decrease concentration of oxygen in the medical air when the blood oxygen concentration of the patient is above a predefined threshold. Similarly, the motorized blender may increase the concentration of the oxygen in the medical air when the blood oxygen concentration of the patient is below the predefined threshold. In some embodiments, the blender may be associated with a filter to filter out impurities, microbes, unwanted particulates, and entrained oils from the medical air. The flow control unit (20) also includes a second flow meter (40) adapted to provide a second predefined volume of the medical air supplied by the blender. In one embodiment, the first flow meter (30) and the second flowmeter may include, but not limited to, a hot wire air meter, a vane meter, a cup anemometer, a pitot tube meter and the like.

Further, the apparatus (10) also includes an inspiratory unit (50) mechanically coupled to the flow control unit (20). In one embodiment, the inspiratory unit (50) may include, but not limited to, three way electromechanical valves such as a solenoid valve, a stepper motor controlled valve and a servo controlled valve. The inspiratory unit (50) includes a first valve (60) mechanically coupled to the first flow meter (30). The first valve (60) is adapted to provide the first predefined volume of the medical air provided by the first flow meter (30) to a patient (80) through a first outlet (70) to assist an inspiration of the patient (80). In a specific embodiment, the first outlet (70) may be associated with a humidifier (180) to humidify the medical air. In such an embodiment, a heat exchanger associated with the humidifier (180) may heat the medical air to a predefined temperature.

Furthermore, in one embodiment, the patient (80) may include, but not limited to, a neonate, an adult, and the like. In some embodiments, the first outlet (70) may provide the medical air to the patient (80) via a nasal canula, or a face mask. The inspiratory unit (50) also includes a second valve (90) mechanically coupled to the second flow meter (40). The second valve (90) is adapted to provide the second predefined volume of the medical air provided by the second flowmeter to the patient (80) through the first outlet (70) to assist an expiration of the patient (80). In one embodiment, the first flow meter (30), the second flow meter (40), and the inspiratory unit (50) may be adapted to be replaced by a motorized flow meter. In such an embodiment, the motorized flow meter may be adapted to provide different volumes of the medical air as per a requirement.

Also, the apparatus (10) further includes an expiratory unit (100) mechanically coupled to the inspiratory unit (50). In one embodiment, the expiratory unit (100) may include, but not limited to, three way electromechanical valves such as a solenoid valve, a stepper motor controlled valve and a servo controlled valve. The expiratory unit (100) is adapted to generate a peak inspiratory pressure during the inspiration of the patient (80) by expelling the first predefined volume of the medical air to a liquid medium (130) at a first depth through a first inlet (110), and a third valve (120). The expiratory unit (100) is also adapted generate a peak expiratory pressure during the expiration of the patient (80) by expelling the second predefined volume of the medical air to the liquid medium (130) at a second depth through the first inlet (110), and a fourth valve (140). In one embodiment, the first depth may be greater than the second depth to provide more pressure during the inspiration.

Additionally, in one embodiment, the third valve (120) and the fourth valve (140) may be interfaced with the liquid medium (130) through a first tube (160) and a second tube (170) respectively. In such an embodiment, the first tube (160), the second tube (170), and the expiration unit (100) may be adapted to be replaced by a tube comprising a plurality of electromechanical valves. In some embodiments, depth of the first tube (160) and the second tube (170) may be adjusted by means of an actuating mechanism. In one embodiment, the first valve (60) and the third valve (120) may be operated simultaneously, and the second valve (90) and the fourth valve (140) may be operated simultaneously. In some embodiments, operation of the second valve (90) and fourth valve (140) may be out of phase with the operation of the first valve (60) and third valve (120). In detail, the second valve (90) and the fourth valve (140) may be closed when the first valve (60) and the third valve (120) are open.

Moreover, in one embodiment, the first tube (160) and the second tube (170) may include pressure manometers to display the pressure of the medical air. In one embodiment, the filter may be placed at a downstream side of the expiratory unit (100) to remove infectious nasal discharges of the patient (80). In one embodiment, unidirectional valves may be used along with the first valve (60), the second valve (90), the third valve (120), and the fourth valve (140) to ensure unidirectional flow of the medical air. In some embodiments, tubings for enabling circulation of the medical air may be associated with pop off valves to safely regulate the pressure of the medical air. In one embodiment, the third valve (120) and the fourth valve (140) may be replaced with a second outlet. In such an embodiment, the expiratory unit (100) may be interfaced with the liquid medium (130) by connecting the first tube (160) between the second outlet and the liquid medium (130) and the first tube (160) may include a pressure pop up valve adapted to operate in one or more set pressures for one or more durations to generate the peak inspiratory pressure and the peak expiratory pressure.

Also, the apparatus (10) further includes a control unit (150) operatively coupled to the expiratory unit (100) and the inspiratory unit (50). The control unit (150) is adapted to provide one or more control signals to operate the first valve (60), the second valve (90), the third valve (120) and the fourth valve (140) to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient (80). In one embodiment, the plurality of parameters may include, but not limited, to at least one of an inspiratory time, an expiratory time, a pressure of the medical air. In some embodiment, the control unit (150) may be adapted to provide the one or more control signals to the first flow meter (30) and the second flow meter (40) to increase flow rate of the medical air upon detecting an irregularity in the pressure of the medical air. In such an embodiment, a proportional integral derivative controller associated with the control unit (150) may control the flow rate of the medical air to compensate the irregularity.

Additionally, in one embodiment, the apparatus (10) may include a pressure sensor associated with the control unit (150). In such an embodiment, the pressure sensor is adapted to sense the pressure of the medical air. In one embodiment, the pressure sensor may be adapted to provide alerts when the pressure sensed is different upon comparing with a predefined threshold. In one embodiment, a user interface associated with the control unit (150) may be capable of displaying a plurality of parameters such as vital parameters of the patient (80), pressure of the medical air, breathing pattern of the patient (80), and respiratory rate of the patient (80). In such an embodiment, the user interface may provide one or more alerts when the plurality of parameters are breaching a predefined range. In one embodiment, the control unit (150) may be operated locally or remotely.

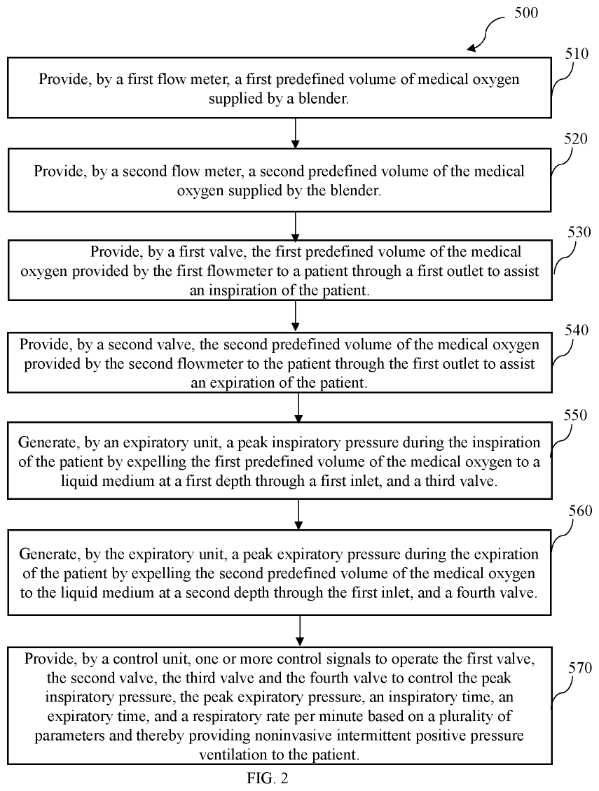

FIG. 2 is a flow chart representing the steps involved in a method (500) for providing noninvasive intermittent positive pressure ventilation in accordance with an embodiment of the present disclosure. The method (500) includes providing a first predefined volume of medical air supplied by a blender in step 510. In one embodiment, providing a first predefined volume of medical air supplied by a blender includes providing a first predefined volume of medical air supplied by a blender by a first flow meter. In one embodiment, the blender may include a motorized blender adapted to provide the medical air to a patient in a concentration range of 21% to 100% through a feedback mechanism based on a blood oxygen concentration of the patient. In detail, the motorized blender may decrease concentration of oxygen in the medical air when the blood oxygen concentration of the patient is above a predefined threshold. Similarly, the motorized blender may increase the concentration of the oxygen in the medical air when the blood oxygen concentration of the patient is below the predefined threshold. In some embodiments, the blender may be associated with a filter to filter out impurities, microbes, unwanted particulates, and entrained oils from the medical air.

The method (500) also includes providing a second predefined volume of the medical air supplied by the blender in step 520. In one embodiment, providing a second predefined volume of the medical air supplied by the blender includes providing a second predefined volume of the medical air supplied by the blender by a second flow meter. In one embodiment, the first flow meter and the second flowmeter may include, but not limited to, a hot wire air meter, a vane meter, a cup anemometer, a pitot tube meter and the like.

The method (500) also includes providing the first predefined volume of the medical air provided by the first flowmeter to a patient through a first outlet to assist an inspiration of the patient in step 530. In one embodiment, includes providing the first predefined volume of the medical air provided by the first flowmeter to a patient through a first outlet to assist an inspiration of the patient includes providing the first predefined volume of the medical air provided by the first flowmeter to a patient through a first outlet to assist an inspiration of the patient by a first valve. In a specific embodiment, the first outlet may be associated with a humidifier to humidify the medical air. In such an embodiment, a heat exchanger associated with the humidifier may heat the medical air to a predefined temperature. In one embodiment, the patient may include, but not limited to, a neonate, an adult, and the like. In some embodiments, the first outlet may provide the medical air to the patient via a nasal canula, or a face mask.

The method (500) also includes providing the second predefined volume of the medical air provided by the second flowmeter to the patient through the first outlet to assist an expiration of the patient in step 540. In one embodiment, providing the second predefined volume of the medical air provided by the second flowmeter to the patient through the first outlet to assist an expiration of the patient includes providing the second predefined volume of the medical air provided by the second flowmeter to the patient through the first outlet to assist an expiration of the patient by a second valve. In one embodiment, the first flow meter, the second flow meter, and the inspiration unit may be adapted to be replaced by a motorized flow meter. In such an embodiment, the motorized flow meter may be adapted to provide different volumes of the medical air as per a requirement.

The method (500) also includes generating a peak inspiratory pressure during the inspiration of the patient by expelling the first predefined volume of the medical air to a liquid medium at a first depth through a first inlet, and a third valve in step 550. In one embodiment, generating a peak inspiratory pressure during the inspiration of the patient by expelling the first predefined volume of the medical air to a liquid medium at a first depth through a first inlet, and a third valve includes generating a peak inspiratory pressure during the inspiration of the patient by expelling the first predefined volume of the medical air to a liquid medium at a first depth through a first inlet, and a third valve by an expiratory unit. In one embodiment, the expiratory unit may include, but not limited to, three way electromechanical valves such as a solenoid valve, a stepper motor controlled valve and a servo controlled valve.

The method (500) also includes generating a peak expiratory pressure during the expiration of the patient by expelling the second predefined volume of the medical air to the liquid medium at a second depth through the first inlet, and a fourth valve in step 560. In one embodiment, generating a peak expiratory pressure during the expiration of the patient by expelling the second predefined volume of the medical air to the liquid medium at a second depth through the first inlet, and a fourth valve includes generating a peak expiratory pressure during the expiration of the patient by expelling the second predefined volume of the medical air to the liquid medium at a second depth through the first inlet, and a fourth valve by the expiratory unit.

Further, in one embodiment, the first depth may be greater than the second depth to provide more pressure during the inspiration. In one embodiment, the third valve and the fourth valve may be interfaced with the liquid medium through a first tube and a second tube respectively. In such an embodiment, the first tube, the second tube, and the expiration unit may be adapted to be replaced by a tube comprising a plurality of electromechanical valves. In some embodiments, depth of the first tube and the second tube may be adjusted by means of an actuating mechanism. In one embodiment, the first valve and the third valve may be operated simultaneously, and the second valve and the fourth valve may be operated simultaneously. In some embodiments, operation of the first valve and the third valve may be out of phase with the operation of the second valve and the fourth valve. In detail, the first valve and the third valve may be closed when the second valve and the fourth valve are open and vice versa.

Furthermore, in one embodiment, the first tube and the second tube may include pressure manometers to display the pressure of the medical air. In one embodiment, the filter may be placed at a downstream side of the expiratory unit to remove infectious nasal discharges of the patient. In one embodiment, unidirectional valves may be used along with the first valve, the second valve, the third valve, and the fourth valve to ensure unidirectional flow of the medical air. In some embodiments, tubings for enabling circulation of the medical air may be associated with pop off valves to safely regulate the pressure of the medical air. In one embodiment, the third valve and the fourth valve may be replaced with a second outlet. In such an embodiment, the expiratory unit may be interfaced with the liquid medium by connecting the first tube between the second outlet and the liquid medium and the first tube may include a pressure pop up valve adapted to operate in one or more set pressures for one or more durations to generate the peak inspiratory pressure and the peak expiratory pressure.

The method (500) further includes providing one or more control signals to operate the first valve, the second valve, the third valve and the fourth valve to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient in step 570. In one embodiment, providing one or more control signals to operate the first valve, the second valve, the third valve and the fourth valve to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient includes providing one or more control signals to operate the first valve, the second valve, the third valve and the fourth valve to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient by a control unit.

Further, in one embodiment, the plurality of parameters may include, but not limited to, at least one of an inspiratory time, an expiratory time, a pressure of the medical air. In some embodiment, the control unit may be adapted to provide the one or more control signals to the first flow meter and the second flow meter to increase flow rate of the medical air upon detecting an irregularity in the pressure of the medical air. In such an embodiment, a proportional integral derivative controller associated with the control unit may control the flow rate of the medical air to compensate the irregularity. In one embodiment, the apparatus may include a pressure sensor associated with the control unit. In such an embodiment, the pressure sensor is adapted to sense the pressure of the medical air.

Furthermore, in one embodiment, the pressure sensor may be adapted to provide alerts when the pressure sensed is different upon comparing with a predefined threshold. In one embodiment, a user interface associated with the control unit may be capable of displaying a plurality of parameters such as vital parameters of the patient, pressure of the medical air, breathing pattern of the patient, and respiratory rate of the patient. In such an embodiment, the user interface may provide one or more alerts when the plurality of parameters are breaching a predefined range. In one embodiment, the control unit may be operated locally or remotely.

Various embodiments of the apparatus and method for providing noninvasive intermittent positive pressure ventilation described above enable various advantages. The first valve, the second valve, the third valve, and the fourth valve enable provision of the medical air in different pressures during the inspiration and expiration of the patient thereby eliminating difficulty during the expiration. Provision of the motorized blender may eliminate a need of human intervention for providing the medical air in concentration ranges of 21% and 100%. Provision of the unidirectional valves ensures unidirectional flow of the medical air. Also, provision of the pop of valves keeps the pressure of the medical air within a predefined limit thereby ensures safety of the patient. Further, the control unit is capable of being operated locally or remotely thereby enabling operational flexibility of the apparatus. The user interface provides real time representation of the vital parameters associated with the patient thereby assisting medical professionals to assess medical conditions of the patient in an effortless manner.

It will be understood by those skilled in the art that the foregoing general description and the following detailed description are exemplary and explanatory of the disclosure and are not intended to be restrictive thereof. While specific language has been used to describe the disclosure, any limitations arising on account of the same are not intended.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all the acts need to be necessarily performed. Also, those acts that are not dependent on other acts may be performed in parallel with the other acts. The scope of embodiments is by no means limited by these specific examples.

We claim:

1. An apparatus (10) for providing noninvasive intermittent positive pressure ventilation comprising:
a flow control unit (20) mechanically coupled to a blender, wherein the flow control unit (20) comprises:
a first flow meter (30) adapted to provide a first predefined volume of medical air supplied by the blender;
a second flow meter (40) adapted to provide a second predefined volume of the medical air supplied by the blender;
an inspiratory unit (50) mechanically coupled to the flow control unit (20), wherein the inspiratory unit (50) comprises:
a first valve (60) mechanically coupled to the first flowmeter, wherein the first valve (60) is adapted to provide the first predefined volume of the medical air provided by the first flow meter (30) to a patient through a first outlet (70) to assist an inspiration of the patient (80);
a second valve (90) mechanically coupled to the second flow meter (40), wherein the second valve (90) is adapted to provide the second predefined volume of the medical air provided by the second flow meter (40) to the patient (80) through the first outlet (70) to assist an expiration of the patient (80);
an expiratory unit (100) mechanically coupled to the inspiratory unit (50), wherein the expiratory unit (100) is adapted to:
generate a peak inspiratory pressure during the inspiration of the patient (80) by expelling the first predefined volume of the medical air to a liquid medium (130) at a first depth through a first inlet (110), and a third valve (120);
generate a peak expiratory pressure during the expiration of the patient (80) by expelling the second predefined volume of the medical air to the liquid medium (130) at a second depth through the first inlet (110), and a fourth valve (140); and
a control unit (150) operatively coupled to the expiratory unit (100) and the inspiratory unit (50), wherein the control unit (150) is adapted to provide one or more control signals to operate the first valve (60), the second valve (90), the third valve (120) and the fourth valve (140) to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient (80).

2. The apparatus (10) as claimed in claim 1, wherein the blender comprises a motorized blender adapted to provide the medical air to the patient (80) in a concentration range of 21% to 100% through a feedback mechanism based on blood oxygen concentration of the patient.

3. The apparatus (10) as claimed in claim 1, wherein the inspiratory unit (50) and the expiratory unit (100) comprises three way electromechanical valves, wherein the three way electromechanical valves comprises at least one of a solenoid valve, a stepper motor controlled valve and a servo controlled valve.

4. The apparatus (10) as claimed in claim 1, wherein the first flow meter (30), the second flow meter (40), and the inspiratory unit (50) are adapted to be replaced by a motorized flow meter.

5. The apparatus (10) as claimed in claim 1, wherein the third valve (120) and the fourth valve (140) are interfaced with the liquid medium (130) through a first tube (160) and a second tube (170) respectively, wherein the first tube (160), the second tube (170), and the expiration unit are adapted to be replaced by a tube comprising a plurality of electromechanical valves.

6. The apparatus (10) as claimed in claim 1, wherein the first outlet (70) is associated with a humidifier (180) to humidify the medical air.

7. The apparatus (10) as claimed in claim 1, wherein the control unit (150) is adapted to provide the one or more control signals to the first flow meter (30) and the second flow meter (40) to increase flow rate of the medical air upon detecting an irregularity in the pressure of the medical air.

8. The apparatus (10) as claimed in claim 1, wherein the plurality of parameters comprises at least one of an inspiratory time, an expiratory time, a pressure of the medical air.

9. The apparatus (10) as claimed in claim 1, comprising a pressure sensor associated with the control unit (150), wherein the pressure sensor is adapted to sense the pressure of the medical air, wherein the pressure sensor is adapted to provide alerts when the pressure sensed is different upon comparing with a predefined threshold.

10. A method (500) comprising:
providing, by a first flow meter, a first predefined volume of medical air supplied by a blender; (510)
providing, by a second flow meter, a second predefined volume of the medical air supplied by the blender; (520)
providing, by a first valve, the first predefined volume of the medical air provided by the first flowmeter to a patient through a first outlet to assist an inspiration of the patient; (530)
providing, by a second valve, the second predefined volume of the medical air provided by the second flowmeter to the patient through the first outlet to assist an expiration of the patient; (540)
generating, by an expiratory unit, a peak inspiratory pressure during the inspiration of the patient by expelling the first predefined volume of the medical air to a liquid medium at a first depth through a first inlet, and a third valve; (550)
generating, by the expiratory unit, a peak expiratory pressure during the expiration of the patient by expelling the second predefined volume of the medical air to the liquid medium at a second depth through the first inlet, and a fourth valve; (560) and
providing, by a control unit, one or more control signals to operate the first valve, the second valve, the third valve and the fourth valve to control the peak inspiratory pressure, the peak expiratory pressure, an inspiratory time, an expiratory time, and a respiratory rate per minute, based on a plurality of parameters and thereby providing noninvasive intermittent positive pressure ventilation to the patient. (570)
